(19) United States
(12) Patent Application Publication
(10) Pub. No.: US 2014/0171930 A1
(43) Pub. Date: Jun. 19, 2014

(54) TISSUE REINFORCING COMPOSITIONS, DEVICES, AND METHODS OF USE

(71) Applicant: Empire Technology Development LLC, Wilmington, DE (US)

(72) Inventor: Junichi Shimada, Kyoto (JP)

(73) Assignee: Empire Technology Development LLC, Wilmington, DE (US)

(21) Appl. No.: 14/238,815

(22) PCT Filed: Feb. 1, 2013
(86) PCT No.: PCT/US13/24340

§ 371 Date: Feb. 13, 2014

Related U.S. Application Data
(60) Provisional application No. 61/595,325, filed on Feb. 6, 2012.

(51) Int. Cl. A61L 31/08; A61L 31/00; A61L 31/04; A61B 18/04

(52) U.S. Cl. CPC A61L 31/08; A61B 18/04; A61L 31/00; A61L 31/042; A61L 31/046
USPC 606/27; 606/40; 606/169

(57) ABSTRACT

Methods for manipulating and/or reinforcing tissues are provided. The method includes applying a tissue reinforcement material to at least a portion of tissue to be manipulated and applying energy to one or both of the tissue reinforcement material and the tissue.

### FIELD

The present application relates generally to tissue reinforcement materials, devices, and methods of use.

### BACKGROUND

Tissue excision and hemostasis are traditionally achieved by manually suturing tissue to be excised, for example by performing double-ligation with silk thread. Mechanical ligation using a stapler is another traditional method for tissue excision.

More recently, energy devices, such as the Harmonic Ace® EnSeal®, and LigaSure® devices have been used to perform tissue excision. The Harmonic Ace®, performs excision of tissue and coagulation of tissue simultaneously. Such devices use energy, such as electricity or ultrasonic waves, instead of performing physical excision with a metal instrument (scalpel). For example, the Harmonic Ace® converts electric energy to mechanical energy in the form of ultrasonic vibrations, causing a blade to vibrate at a frequency of 55,500 Hz and amplitude of 50 to 100 μm in the direction of the longitudinal axis. An incision is made by repeatedly moving the tissue beyond the elastic limit of the tissue using a vibrating blade. The coagulation involves using vibrations to denaturize protein to generate a viscous coagulant that sutures or welds a blood vessel. Frictional heat is generated in the tissue, causing closure and fusion of the tissue.

### SUMMARY

In some embodiments, a method for treating and/or manipulating tissue is provided. The method includes applying a tissue reinforcement material to at least a portion of a tissue to be treated. The method further includes applying energy to the tissue reinforcement material, the tissue, or both the tissue reinforcement material and the tissue, wherein applying energy causes fusion between at least a part of the tissue reinforcement material and at least a part of the tissue. The method further includes manipulating the tissue.

In some embodiments, a composition for reinforcing tissue is provided. The composition includes a biocompatible material and a lipid. In some embodiments, the biocompatible material is a sheet, gel, or both a sheet and a gel that can be applied to a surface of a tissue, and wherein the lipid is associated with the biocompatible material.

In some embodiments, a device for treating tissue is provided. The device can include an energy delivery head configured to impart energy to a tissue reinforcement material, a tissue, or both the tissue reinforcement material and the tissue. The device further includes a structure configured to support the tissue reinforcement material.

In some embodiments, a kit for treating tissue is provided. The kit includes a first device configured to impart energy to a tissue to be treated, a second device configured to impart energy to a tissue reinforcement material, and a tissue reinforcement material.

In some embodiments, a method for treating tissue is provided. The method includes applying a tissue reinforcement material including a lipid to at least a portion of a tissue to be treated. The method further includes applying energy to the tissue reinforcement material, the tissue, or both the tissue reinforcement material and the tissue, wherein applying energy causes fusion between at least a part of the tissue reinforcement material and at least a part of the tissue.

In some embodiments, a hemoclip is provided. The hemoclip includes a malleable clip that includes lecithin.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 depicts a flow chart outlining some embodiments for manipulating a tissue.

FIGS. 2A-2C are drawings depicting a side view of a tissue undergoing a method of treating according to some embodiments.

FIGS. 3A is a photograph and 3B is a drawing depicting various views of a method of treating a tissue according to some embodiments.

FIG. 4 is a flow chart depicting a method of treating a tissue according to some embodiments.

FIG. 5 is a flow chart depicting a method of treating a tissue according to some embodiments.

FIG. 6 is a depiction of two graphs displaying results regarding the time required for excision and pressure resistance for a vein treated with a tissue reinforcement material.

FIG. 7 is a depiction of graph displaying results regarding the time required for excision and pressure resistance for a vein treated with a tissue reinforcement material.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

In some embodiments, a method of using a tissue reinforcement material on various tissue surfaces, such as blood vessels, is provided. In some embodiments, the method can be employed on a tissue to be excised or otherwise manipulated in conjunction with the use of energy to coagulate blood. In some embodiments, the tissue reinforcement material can be applied to or wrapped around a tissue (e.g., a blood vessel) to be treated. A device can be used to apply the tissue reinforcement material to the tissue. Energy can be applied to the tissue or the tissue reinforcement material or both (optionally through the same device or a different device). In some embodiments, the application of energy fuses at least a part of the tissue reinforcement material to at least a part of the tissue. In some embodiments, following the fusion, the tissue can be manipulated in any number of ways. In some embodiments, the tissue is manipulated during the fusion itself, for example, the fusion provides added strength to the tissue, as the tissue itself is being cut and/or otherwise manipulated.

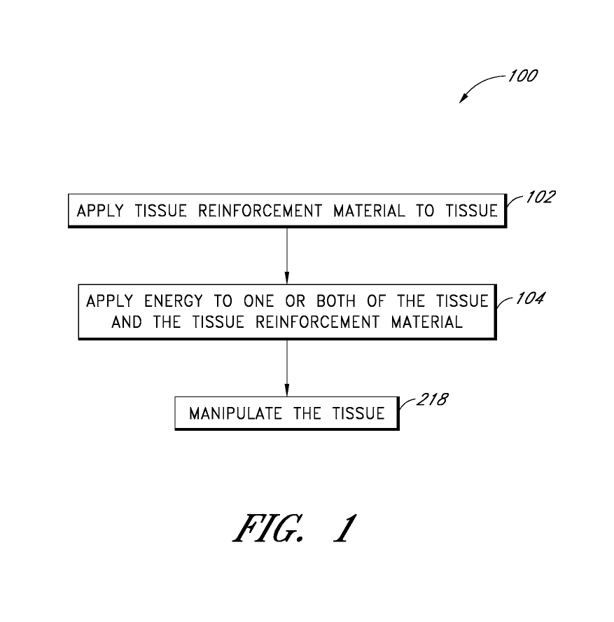

In some embodiments, a method for treating a tissue is provided. Some embodiments of such a method 100 are shown in the flow chart depicted in FIG. 1. In some embodiments, the method includes applying a tissue reinforcement material to at least a portion of a tissue (block 102). In some embodiments, the method further includes applying energy to one or both of the tissue and the tissue reinforcement material (block 104). Applying energy to one or both of the tissue and the tissue reinforcement material can cause fusion between at least a part of the tissue reinforcement material and at least a part of the tissue. In some embodiments, the method further includes manipulating the tissue (block 218). One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In some embodiments, the tissue to be manipulated includes at least one of a blood vessel, a neuron, a surface of an organ, or a muscle. Other tissues can also be manipulated. In some embodiments, any tissue can be manipulated by one or more of the methods provided herein. In some embodiments, one can adjust and/or choose the reinforcement material so that its properties are appropriate for its location and/or use.

In some embodiments, the tissue reinforcement material includes one or more of a protein, a plant based protein, oxidized cellulose, a polyglycolic acid, a fibrinogen, collagen, a lipid, lecithin, lecithin analogs, and combinations thereof. In some embodiments, the tissue reinforcement material is any material that is 1) biologically compatible with the tissue it is to be fused to and 2) fusible with a tissue and/or blood. In some embodiments, the reinforcement material can include Surgicel® material, e.g., an oxidized cellulose polymer of polyanhydroglucuronic acid. In some embodiments, the reinforcement material can include TachoComb® material, e.g., collagen sheets coated with fibrinogen, thrombin, and aprotinin. In some embodiments, the reinforcement material can include Neoveil® material, e.g., an absorbable polyglycolic acid. As noted below, in some embodiments, the reinforcement material can include a lipid (e.g., as part of the material itself and/or as a coating on a base material).

In some embodiments, applying the tissue reinforcement material to at least a portion of the tissue includes wrapping the tissue reinforcement material around a circumference of the tissue. In some embodiments, the tissue reinforcement material is wrapped around the entire circumference of the tissue. In some embodiments, the tissue reinforcement material is wrapped around a portion of the circumference of the tissue, for example, in some embodiments, the tissue reinforcement material is wrapped around about, but not limited to, 10%, 20%, 30%, 40%, 50%, 60%, 70%, 80%, 90%, 95%, 99%, or 100% of the circumference of the tissue. In some embodiments, the tissue reinforcement material can be fully wrapped around the circumference of the tissue, and continues to “overwrap” at least a portion of the tissue, resulting in at least a portion of the tissue with two layers of overlapping tissue reinforcement material. In some embodiments, the tissue reinforcement material is applied to the tissue by placing the tissue reinforcement material on the tissue. In some embodiments, the tissue reinforcement material is adhered to the tissue. In some embodiments, the tissue reinforcement material is applied to the tissue as a single piece. In some embodiments, the tissue reinforcement material is applied to the tissue as multiple pieces. For example, but not limited to, 2, 3, 4, 5, 6, 7, 8, 9, 10, or more pieces of tissue reinforcement material can be used. In some embodiments, the reinforcing material can be of any thickness. In some embodiments, the thickness can depend on the specific properties of the reinforcing material (energy capacity, etc.) or shape of the tip of the energy device used. In some embodiments, the thickness can depend upon the thickness of the tissue to be manipulated and/or the type of manipulation. In some embodiments, the thickness can be greater than 0.0001 mm, e.g., 0.0001, 0.001, 0.01, 0.05, 0.09, 0.1, 0.2, 0.3, 0.4, 0.5, 0.6, 0.7, 0.8, 0.9, 1, 2, 3, 4, 5, 6, 7, 8, 9, 10, 20, or 30 mm, including any range defined between any two of these values and any range above any one of these values.

In some embodiments, applying the tissue reinforcement material can be performed manually. For example the tissue reinforcement material can be manually wrapped around at least a portion of tissue and secured with a medical clip or other suitable clip-like mechanism. In some embodiments, applying the tissue reinforcement material can be performed by a suitable device. In some embodiments, a combination of manual and device-related application is also possible. FIG. 3A depicts some embodiments in which the tissue reinforcement material 16 is placed above and/or below the tissue 12 to be treated and held there by the arms of the energy device 14. In some embodiments, an adhesive layer can be employed to assist in applying the reinforcing material. In some embodiments, the reinforcement material can be provided as a liquid and/or gel and can be painted or spread onto the tissue. In some embodiments, the tissue reinforcement material can be supplied as a liquid and can be injected into and/or onto the tissue. In some embodiments, the reinforcement material can be sprayed onto the tissue. In some embodiments, the reinforcement material is not limited by how it can be applied. In some embodiments, the reinforcement material can be applied in a paste form. In some embodiments, the reinforcement material can be applied via a clip and/or be part of a clip. For example, in some embodiments, a biomaterial clip can be provided that can include at least some amount of a reinforcement material. Application of the clip, allows for the placement of the reinforcement material. In some embodiments, the clip can be a biomaterial and/or reinforcement clip. In some embodiments, the clip can be configured as a hemoclip. In some embodiments, the clip can include both a malleable metal section, and an interior section, which is to be placed against the tissue, that can be coated with the reinforcement material, such as a lecithin, lecithin component, and/or analog thereof. In some embodiments, the reinforcement material need not include a metal or other, separate rigid and malleable support, but the reinforcement material itself can be adequately malleable so as to be adequate as a temporary clip (as the clip need only stay in place long enough for the application of energy). In some embodiments, a coating of the reinforcement material is sprayed onto the clip. In some embodiments, the clip includes a layer of the reinforcement material. In some embodiments, the clip is a hemoclip that is made purely from the reinforcement material. In some embodiments, the clip is a hemoclip that includes a metal clip so as to allow the application of heat to the metal, to heat the biomaterial (such is lecithin) in a more focused manner.

Various forms of energy can be used to perform various embodiments provided herein. In some embodiments, applying energy to the device includes altering a temperature of the tissue reinforcement material. For example, heat can be applied to the tissue reinforcement material. In some embodiments, the heat can be generated by any number of ways, for example, radiation, chemical, electrical (resistive), vibration (such as ultrasound), etc.

Applying energy can also include controlling the type and characteristics of the energy applied. For example, one or both of energy output and energy frequency can be controlled. In some embodiments, the method can involve starting at a first frequency for a part of the fusing process, changing an intensity of the energy after at least part of the fusion (e.g., an increase or decrease) to then allow further manipulation. In some embodiments, the characteristics of the energy being applied stays approximately consistent throughout the process. In some embodiments, the energy level can be increased so as to allow for the manipulation itself, e.g., a cutting of the tissue that has the reinforcing material associated with it. Specific treatment conditions can be changed appropriately in accordance with the properties and thickness of the tissue and the tissue reinforcement material. The energy applied can include at least one of ultrasonic energy, electricity, light, microwave, or radiation. Other types of energy differentials are also contemplated, such as cryoablation.

In some embodiments, fusion of a part of the tissue and a part of the tissue reinforcement material is caused, at least in part, by denaturation of at least a part of the tissue. The denaturation can occur in a protein of the tissue and/or surrounding tissue, such as blood. In some embodiments, at least some of the tissue reinforcement material applied is fused to tissue surrounding the tissue to be treated. In some embodiments, only a fraction of the tissue reinforcement material applied is fused to tissue surrounding the tissue to be treated. In some embodiments, for example, but not limited to, about 5, 10, 20, 30, 40, 50, 60, 70, 80, or 90% of the tissue reinforcement material applied is fused to tissue surrounding the tissue to be treated. In some embodiments, the fusion between a part of the tissue reinforcement material and at least a part of the tissue causes hemostasis. In some embodiments, the fusion causes at least partial hemostasis (e.g., reducing blood flow by up to about 1, 2, 3, 4, 5, 10, 20, 30, 40, 50, 60, 70, 80, 90, 95, 98, 99, or 100%). Specific conditions can be appropriately selected in accordance with the properties and thickness of the tissue, the tissue reinforcement material, and the purpose of the manipulation applied to it. In some embodiments, the fusion is adequate to reduce and/or stop blood flow in dissections and suturing. In some embodiments, the fusion is adequate as long as blood stops.

In some embodiments, manipulating the tissue includes cutting the tissue. In some embodiments, manipulating the tissue includes excision of tissue. In some embodiments, manipulating the tissue includes moving the tissue. In some embodiments, manipulating the tissue includes fusing a first tissue to a second tissue. Other manipulations are also possible. For example, in some embodiments, manipulating the tissue includes ligation. In some embodiments, manipulating the tissue includes (but is not limited to), moving, cutting, sealing, fusing, expanding, restricting, and/or cauterizing the tissue.

In some embodiments, manipulating the tissue is performed by a device configured to impart energy to the tissue. For example, the energy imparted can be heat, electricity, or radiation. Other types of energy (e.g., as discussed herein) are also possible. The device can be configured to allow control of the characteristics (e.g., frequency, driving time, and/or output) of the energy imparted.

In some embodiments fusion occurs concurrently with the cutting of the tissue (e.g., such as when one cuts and/or ligates a tissue such as a vein). In some embodiments, fusion commences just before the commencement of cutting the tissue. In some embodiments, fusion continues until after cutting the tissue. In some embodiments, fusion can commence and conclude prior to the cutting or other manipulation of the tissue. It will be appreciated that the timing of the fusion described herein can be applied to embodiments in which manipulation of the tissue includes something other than cutting of the tissue (such as any of the other options provided herein or known to one of skill in the art).

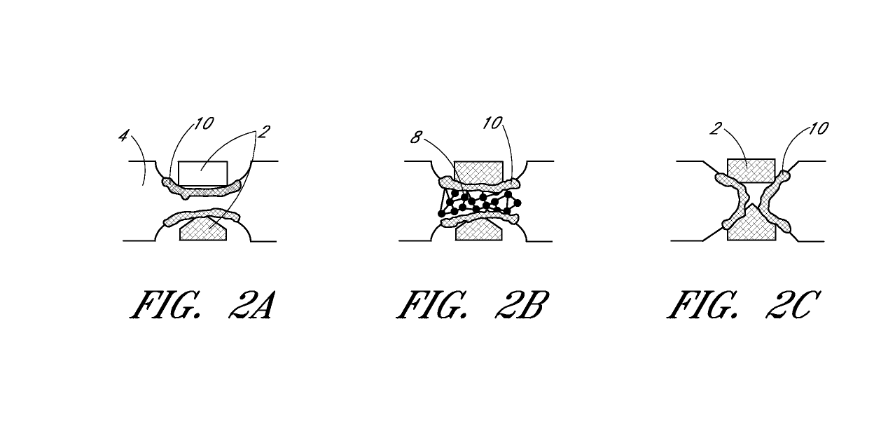

FIGS. 2A-2C depict some embodiments of a method of treating tissue. FIG. 2A depicts a side view of a blood vessel 4 with tissue reinforcement material 10 wrapped around the blood vessel 4. An energy device 2 is shown clamping down on the tissue 4 and the tissue reinforcement material 10. As the energy device 2 imparts energy (e.g., ultrasonic energy) to the tissue 4 and/or tissue reinforcement material, the proteins in the cells of the tissue can start to denature as shown in FIG. 2B and, optionally, form a viscous coagulum 8 (in some embodiments, no coagulum is formed, and the energy simply fuses the reinforcing material to the tissue). In some embodiments, additional frictional heat can cause closure and fusion of the vascular wall (FIG. 2C).

FIGS. 3A and 3B show some embodiments of treating tissue 12 using a device such as the Harmonic Ace® energy device 14. As shown in FIG. 3A, the tissue reinforcement material 16 is placed above and below the tissue 12 to be treated. The energy device 14 then applies energy to the tissue reinforcement material, simultaneously fusing it to at least some of the circumference of the tissue 12 and cutting through the fused tissue and reinforcement material. FIG. 3B depicts the tissue 12 after treatment. The reinforcement material 16 is fused to the ends or stumps of the treated tissue. The tissue 18 near the ends or stumps is shown to be thicker and darker, representing the increased strength caused by the coagulation of the reinforcement material and the tissue.

FIG. 4 is a flow chart depicting a method 200 for manipulating a tissue. The method includes wrapping, using a suitable device, a tissue reinforcement material including at least one of an oxidized cellulose, a polyglycolic acid, a fibrinogen, a collagen, lecithin, lecithin analogs, or a lipid around at least a portion of a circumference of the tissue to be treated (block 202). The method can further include applying energy, using a device configured to impart energy, to the tissue or tissue reinforcement material, or both. Applying energy causes at least some fusion between at least a part of the tissue reinforcement material and at least a part of the tissue (block 204). The method further includes manipulating the tissue (block 206).

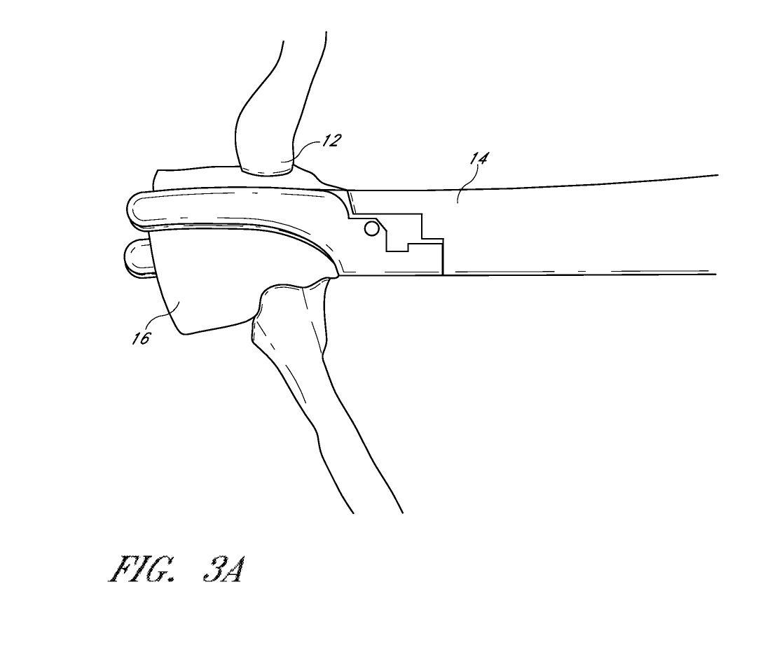

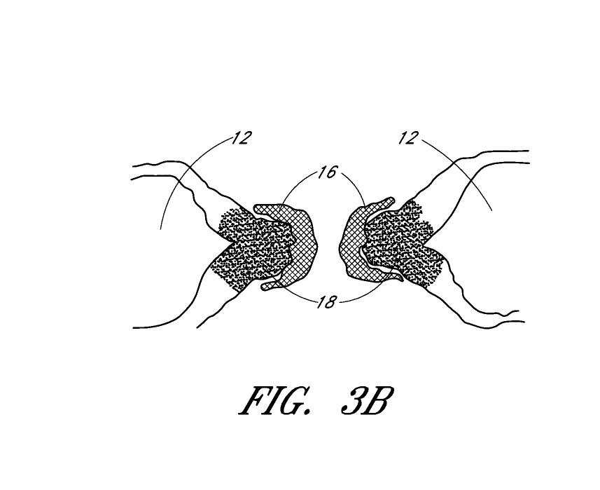

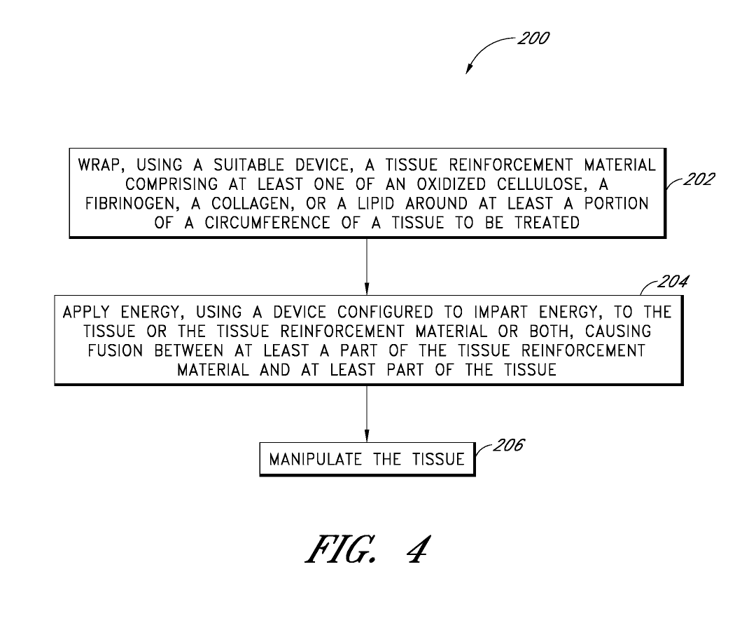

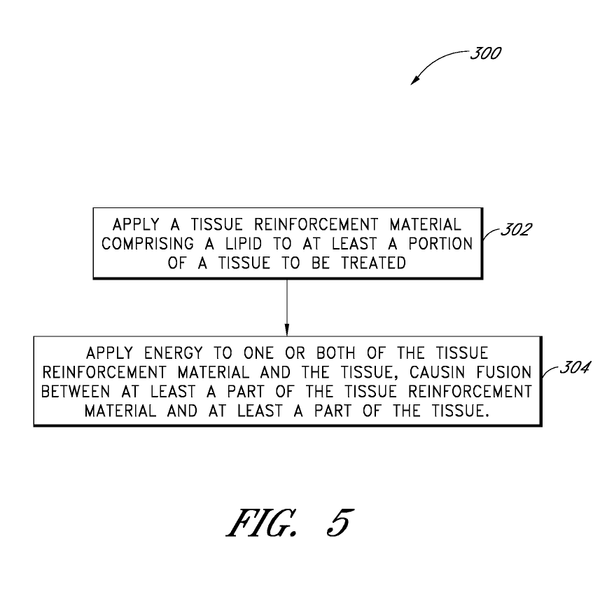

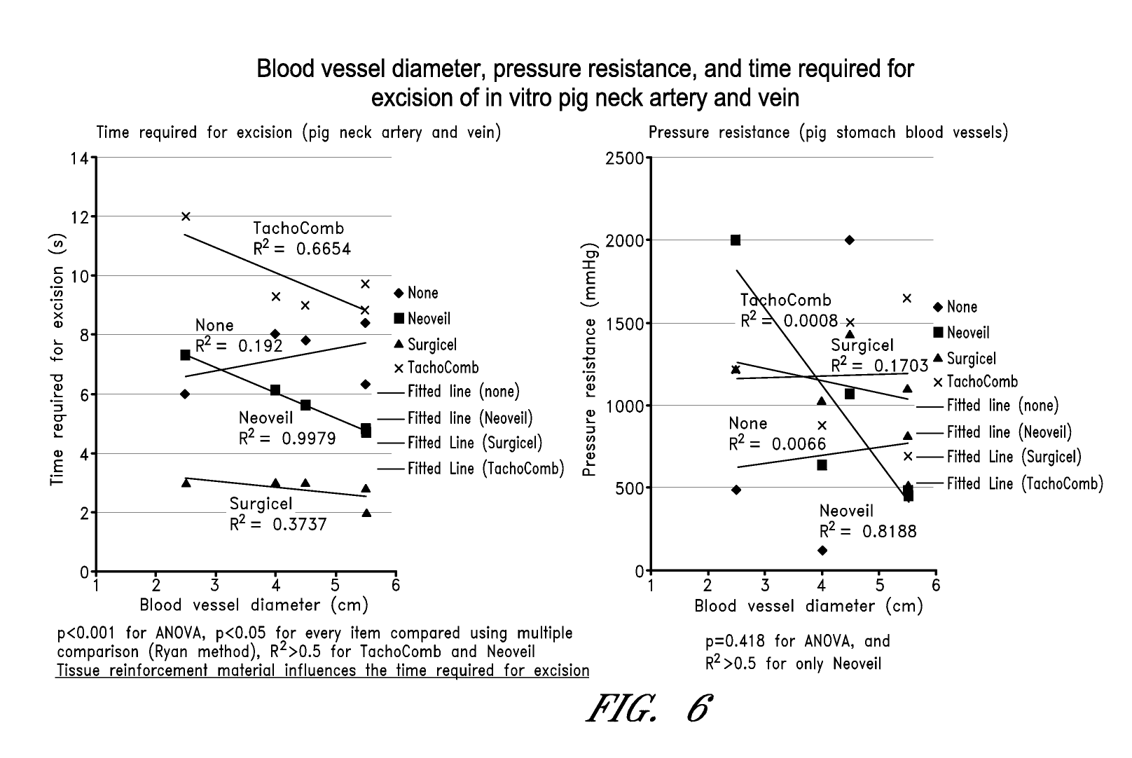

FIG. 5 depicts some embodiments of a method 300 for treating a tissue. The method 300 includes applying a tissue reinforcement material including a lipid to at least a portion of tissue to be treated (block 302). The method further includes applying energy to one or both of the tissue reinforcement material and the tissue, causing fusion between at least a part of the tissue reinforcement material and at least a part of the tissue (block 304).

In some embodiments, by fusing the tissue and the tissue reinforcement material in the manner described herein, the at least a part of the tissue structure can be reinforced and can provide improved pressure resistance during manipulation of the tissue. Furthermore, the fusion of the tissue and the tissue reinforcement material also increases the thickness of the coagulum formed by the tissue and the material. In some embodiments, increased thickness of the coagulum can improve excision precision of the energy devices described herein.

In some embodiments, a composition for reinforcing a tissue is provided. In some embodiments, the composition includes a biocompatible material. In some embodiments, the reinforcing material can also include a lipid (although this is not required for all embodiments). In some embodiments, the reinforcing material is protein based. In some embodiments, the reinforcement material includes at least one of a lipid or a protein. In some embodiments, the reinforcement material can be formulated as a sheet, wrap, and/or solid, so that it can be wrapped around a tissue. In some embodiments, the reinforcement material can be formulated as a paste, gel, and/or solution, so that it can be spread on top of and/or injected into a tissue. In some embodiments, the reinforcement material can be provided in any state (e.g., solid, liquid, gel, aerosolized, etc.). When in a paste embodiment, the paste can be readily spread onto a surface of a tissue to be manipulated. In some embodiments, the paste can be placed onto an arm of a device configured to impart energy to a tissue to be treated and/or to the reinforcement material.

In some embodiments, a lipid is associated with the biocompatible material. In some embodiments, the lipid includes a lipid found in the subject whose tissue is being manipulated. In some embodiments, the lipid can include one or more of a fat, wax, sterol, fat-soluble vitamin, monoglyceride, diglyceride, triglyceride, phospholipid, or others. In some embodiments, the lipid can include one or more of a hydrophobic lipid or an amphiphilic lipid. In some embodiments, the lipid can include one or more of a fatty acyl, a glycerolipid, a glycerophospholipids, a sphingolipid, a saccharolipid, and/or a polyketide. In some embodiments, the lipid can include one or more of a phosphatidylcholine, a phosphatidylethanolamine (PE or GPEtn), a phosphatidylserine (PS or GPSer). In some embodiments, any lipid can be employed. Without intending to be limited by theory, it is understood that, because lipids are a major component of cell membranes, mixing a lipid with the biocompatible material can improve the speed and accuracy of the fusion between the tissue and the biocompatible material.

In some embodiments, the reinforcing material includes a lipid. In some embodiments, the reinforcing material includes lecithin, a component thereof, and/or an analogue thereof. In some embodiments, the reinforcement material includes at least one or more of: phosphoric acid, phosphatidylinositol, phosphatidylethanolamine, phospholipids, phosphatidylcholine, triglycerides, choline, fatty acids, glycerol, and/orglycolipids. In some embodiments, the lecithin can include one or more of: phosphoric acid, phosphatidylinositol, phosphatidylethanolamine, phospholipids, phosphatidylcholine, triglycerides, choline, fatty acids, glycerol, and/or glycolipids. In some embodiments, the lecithin can be a hydrolysed lecithin, such as lysophospholipids. In some embodiments, the lecithin can be fractionated. In some embodiments, the lecithin can be from any source. In some embodiments, the lecithin can be from soya. In some embodiments, one or more of the lecithin ingredients can be present in the reinforcing material. In some embodiments, one or more of the lecithin ingredients can be at least 0.001% of the reinforcing material, by weight, for example, 0.001, 0.01, 0.1, 1, 2, 3, 4, 5, 10, 20, 30, 40, 50, 60, 70, 80, 90, 91, 92, 93, 94, 95, 96, 97, 98, 99, 99.9, or 100 percent of the reinforcing material is at least one of the ingredients noted above in regard to lecithin. In some embodiments, the lipid includes a choline moiety, a phosphate group, a glycerol, a saturated fatty acid and/or an unsaturated fatty acid. In some embodiments the lecithin is water soluble. In some embodiments, the lecithin is lipid soluble. In some embodiments, the lecithin, or component thereof, is of a quality high enough for GRAS standards (to be generally regarded as safe). In some embodiments, the lecithin, or component thereof, is at least as pure as to be suitable for pharmaceutical and/or personal care applications. In some embodiments, one or more of the lecithin components is saturated. In some embodiments, one or more of the lecithin components is unsaturated. In some embodiments, the reinforcing material applied to the subject can be any lipid and/or lecithin component or product, so long as it is not native to the subject. Thus, in some embodiments, the lecithin and/or phospholipid is foreign to the host. In some embodiments, the lecithin and/or phospholipid is native to the host; and thus, can have been isolated from the host, treated (for example for enrichment of lecithin components) and applied to a desired location in the host.

In some embodiments, the biocompatible material includes oxidized cellulose, polyglycolic acid, fibrinogen, collagen, a lipid, combinations thereof, and the like. It will be appreciated that surgical hemostatic materials regularly employed in surgery can be used. The biocompatible material can include a protein. A wide variety of biocompatible materials, including those not specifically disclosed herein, are possible. The biocompatible material can be configured into a sheet, a ribbon, a liquid, a sprayable material, a gel, or some combination thereof. In some embodiments the material can form (or be in) a cylindrical shell or tube. In some embodiments, the sheet can form multiple cylindrical shells. For example, the sheet can form two cylindrical shells. In some embodiments, the sheet is configured as a “Y” shape or “V” shape, e.g., two sides that can surround a vein. In some embodiments, the sheet can be configured into a “pants” configuration, so that ends of tweezers or other instruments can be fitted with the sheet and readily be applied to a vein or other curved tissue surface.

In some embodiments, the biocompatible material can be used for promoting healing of tissue. Regenerating factors, such as EGF and VEGF, which promote regeneration after tissue excision, and/or antibodies, which promote curing, combinations thereof, or the like can be mixed into the reinforcement material.

### Devices

In some embodiments, a device for treating tissue and/or manipulating a tissue is provided. In some embodiments, the device includes an energy delivery head configured to impart energy to a tissue reinforcement material, a tissue, or both a tissue reinforcement material and the tissue. The energy imparted can be heat, radiation, light, electricity, cryoablation, ultrasonic energy, combinations thereof (or any other form of energy provided herein). In some embodiments, the device further includes a structure configured to support and/or position the tissue reinforcement material. In some embodiments, the device can be configured to cause fusion between at least a part of the tissue reinforcement material and at least a part of the tissue.

In some embodiments, the energy delivery head includes at least one of an optical output, an ultrasonic head, a heat emitting electrode, a conductor of heat, a conductor of electricity, or a plasma source. In some embodiments, the device is configured to control characteristics of the energy imparted to the tissue. For example, in some embodiments, the device is configured to control a frequency of the energy imparted to the tissue. In some embodiments, the device can be configured to control other characteristics (e.g., output, driving time) of the imparted energy. In some embodiments, the device includes an energy source. In some embodiments, the device merely acts to deliver energy from a different location.

In some embodiments, the structure includes a repository for the placement of the tissue reinforcement material. In some embodiments, the repository can be located at a tip of the device. In some embodiments, the repository is located proximal to the energy delivery head. Other locations for the repository are also possible. The repository can be configured to store tissue reinforcement material of different forms (e.g., gel, sheet).

In some embodiments, the structure includes one or more arms. In some embodiments at least one of the arms is configured to receive (for example) a sleeve of the tissue reinforcement material. The arms of the device can be configured to apply a sleeve of the tissue reinforcement material to the tissue to be treated. In some embodiments, the arms are actuatable to a closed position with respect to one another. Other configurations for the structure are also possible. For example, in some embodiments, the structure includes one or more limbs or hooks configured to apply tissue reinforcement material in the form of a gel to tissue to be treated by secreting the tissue reinforcement material onto the tissue. In some embodiments, the structure includes arms or hooks that can be manipulated in order to apply a sheet of tissue reinforcement material to a tissue to be treated. For example, the arms or hooks are configured to wrap a sheet of tissue reinforcement material around at least a part of a circumference of the tissue to be treated.

In some embodiments, the energy source can include, for example, a commercially available energy source such as a HARMONIC ACE® device, an ENSEAL® device, or a LIGASURE® device.

### Kits

In some embodiments, a kit for treating tissue is provided. In some embodiments, the kit includes a first device configured to impart energy to a tissue to be treated and a second device configured to impart energy to a tissue to a tissue reinforcement material. The kit further includes a tissue reinforcement material. The first device and second device are, in some embodiments, different devices. In some embodiments, the first device and the second device are the same device. In some embodiments, the second device is configured to assist in applying the tissue reinforcement material. In some embodiments, the first and second devices are different parts of the same device. In some embodiments, the first and second devices are the same part of a same device (e.g., when ultrasonic energy is used for both fusing and cutting).

In some embodiments, at least the first device includes a first arm and a second arm situated in a pincher arrangement. In some embodiments, the tissue reinforcement material includes a first sleeve and a second sleeve. In some embodiments, the first sleeve is configured to go over the first arm of the first device and the second sleeve is configured to go over the second arm of the first device. In some embodiments, at least the first device is configured to assist in applying the tissue reinforcement material in a way other than that just described. For example, the device can include a repository to store tissue reinforcement material in the form of a gel and can include a further structure such as limbs or hooks to apply the gel. In some embodiments, at least the first device includes first and second arms actuatable between open and closed positions. In some embodiments, the first and second arms can be used to apply one or more sheets of tissue reinforcement material to tissue to be manipulated. For example, the first and second arms can be used to wrap a sheet of tissue reinforcement material at least partially around a circumference of tissue to be treated. A medical clip or other suitable clipping mechanism can be used to secure the sheet of tissue reinforcement material around the tissue.

### Additional Aspects Relating to Some Embodiments

In some embodiments, the methods, compositions, devices, and kits described herein allow for the safer treatment of a wider range of tissue than do conventional methods. Traditional tissue excision (without the present tissue reinforcement material) performed with energy devices such as those described reduce the amount of damage inflicted on the patient's body; however, when used on a blood vessel, such as the pulmonary artery, sufficient coagulant is not formed because the vascular wall is extremely thin, approximately 200 μm, and thus sufficient hemostasis is not achieved. By fusing the blood vessel to be treated with a tissue reinforcement material prior to treatment, hemostasis can be more easily achieved, even in a blood vessel having a thin vascular wall, on which it is difficult to form a coagulum with the energy transmitted from an energy device, according to the related art.

In addition, although performing coagulation and excision at low temperature reduces thermal damage to the surrounding tissue, it may cause an increase in the time required for the excision. In particular, the vascular wall of the pulmonary artery is prone to tension caused by respiratory and cardiac motion. Therefore, the risk of vascular damage increases as the amount of time required for excision increases. In some embodiments, because the pressure resistance of the tissue to be excised can be improved by fusing tissue reinforcement material to the tissue to be treated, the amount of time required for incision can be reduced. Accordingly, the safety of excision or other manipulation performed by an energy device can be improved by the use of a tissue reinforcement material, and wider application of the energy device according to the present invention can become possible.

In some embodiments, in surgery using an energy device according to a conventional method, unless the conditions, such as temperature, are controlled extremely precisely, nearby healthy tissue, such as nerves, may be severely damaged by the transmitted energy. In contrast, with some of the embodiments provided herein, such damage can be reduced since reinforcement is provided by the tissue reinforcement material, and in addition to reducing the amount of time required for treatment, the safety of the operation can be further improved.

In conventional surgery, hemostasis of thin blood vessels, excision of large tissue, etc. require highly developed skills. For example, ligation of the pulmonary artery using an endoscope is very difficult and requires highly developed skills. Automatic suture instruments are used for ligation of thick blood vessels; however, there have been reports of deaths due to vascular damage caused by misfiring of the device. Furthermore, branching of very thin blood vessels often occurs in the pulmonary artery, and these are ligated with silk thread; however, such ligation bears a high risk of vascular damage and is often mentally demanding. Although some procedures, such as those using endoscopic techniques will generally require high-level skills, some of the present embodiments simplify the techniques, making endoscopic surgery safer and faster and increasing the number of doctors and facilities that can perform the technique. This also reduces a burden of patients taking the surgery.

### EXAMPLES

### Example 1

### In Vitro Examination of the Influence of a Reinforcing Material

A pig neck artery, vein, and stomach blood vessels with diameters of 2.5, 4, 4.5, and 5.5 mm were used to perform tests on the amount of time required for excision and the pressure resistance. Tissue reinforcement materials including oxidized cellulose (Surgicel® material), polyglycolic acid (Neovei®1 material), and fibrinogen (TachoComb® material) were wrapped around the blood vessels to be treated. A control blood vessel without tissue reinforcement material was also used. Excision was performed using the commercially available Harmonic Ace® device. The ultrasonic waves used in excision were set to a vibration frequency of 55.5 kHz. The time required for excision was shortened as compared with the control treatment, regardless of the type of tissue reinforcement material used. In the cases of oxidized cellulose and polyglycolic acid as the tissue reinforcement material, improvement in pressure resistance was also clearly observed.

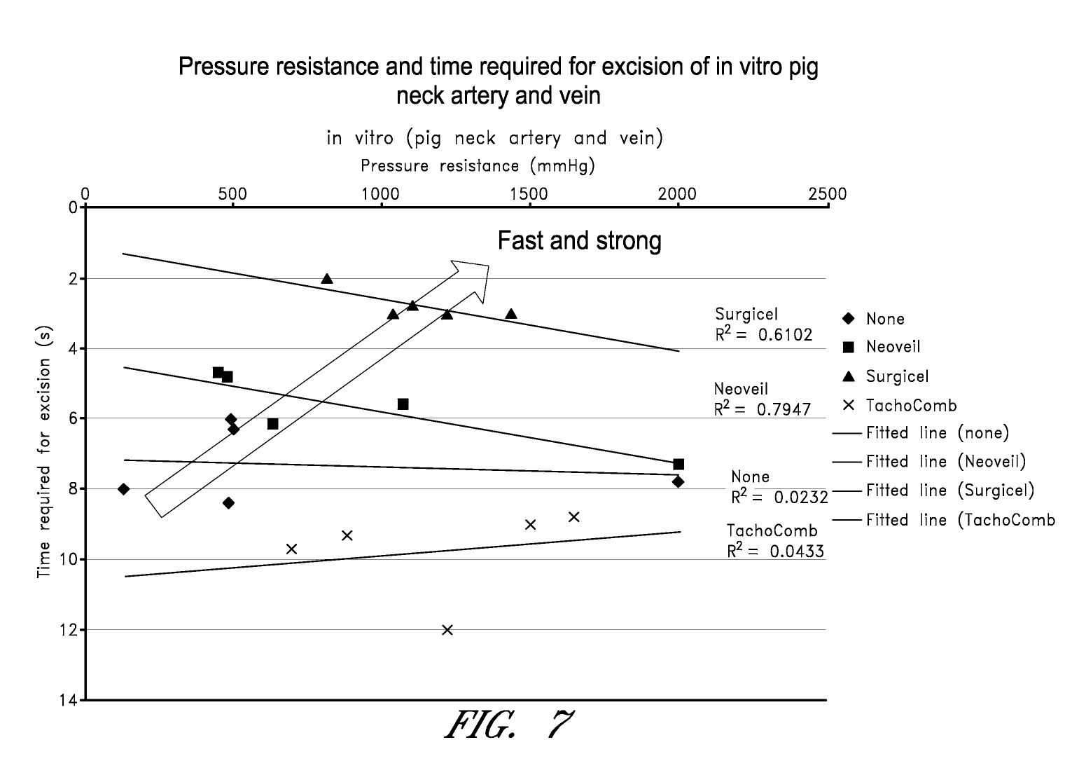

The results are summarized in the graphs in FIGS. 6 and 7. As shown in the results, the various tissue reinforcement materials had varying impacts on the time required for excision and pressure resistance of the manipulated material. Surgicel® material (an oxidized cellulose polymer of polyanhydroglucuronic acid) appeared to have the greatest ability in providing for both a faster excision time and a greater pressure resistance, as compared to TachoComb® material (collagen sheets coated with fibrinogen, thrombin, and aprotinin) and Neoveil material (an absorbable polyglycolic acid). As can be seen in the results in FIGS. 6 and 7, the various materials clearly altered the time required for excision and pressure resistance of the blood vessel. In particular, of the materials tested, the Surgicel® material produce an arrangement where the least amount of time was required for excision and the vein had the best pressure resistance.

### Example 2

### Reinforcement Materials

Composition 1: A reinforcement material including an oxidized cellulose polymer of polyanhydroglucuronic acid is provided and coated with lecithin, thereby providing a reinforcing material that includes a lipid.

Composition 2: A reinforcement material including fibrinogen is provided and coated with a phospholipid, thereby providing a reinforcing material that includes a lipid.

Composition 3: A reinforcement material including an absorbable polyglycolic acid is provided and coated with lecithin, thereby providing a reinforcing material that includes a lipid.

Composition 4: A malleable hemoclip is coated with lecithin and then the lecithin is allowed to at least partially dry, thereby providing a hemoclip that can be applied directly to the site of interest and energy be applied directly to the clip.

### Example 3

### Method of Cutting a Vein with a Lipid Based Reinforcing Material

A tissue reinforcement material from Example 2 is wrapped around a blood vessel in a subject. Fusion of the reinforcement material and excision is performed using a Harmonic Ace® device. The ultrasonic waves are set to a vibration frequency of 55.5 kHz. Ligation/hemostasis is performed while controlling the temperature under predetermined conditions.

The driving time and energy output of the energy device are appropriately adjusted in accordance with the diameter of the blood vessel or the thickness of the vascular wall; however, by reinforcing the blood vessel by wrapping the tissue reinforcement materials around it, higher power treatment in a shorter amount of time will be possible, compared with treatment with a known energy device. Furthermore, the presence of the lipid in the reinforcement material provides for further advantages over embodiments which lack the use of the lipid with the reinforcement material, including at least one of superior fusion speed, superior fusion accuracy, and/or a higher degree of compatibility to the tissue (in comparison to other bio compatible materials which just adhere to the tissue).

### Example 4

### Method of Cutting a Vein with a Lipid Based Reinforcing Material

A sheet of tissue reinforcement material that includes at least 50% lecithin, by weight, is wrapped around a blood vessel in a subject. Fusion of the reinforcement material and excision is performed using a Harmonic Ace® device. The ultrasonic waves are set to a vibration frequency of 65.5 kHz. Ligation/hemostasis is performed while controlling the temperature under predetermined conditions.

The driving time and energy output of the energy device are appropriately adjusted in accordance with the diameter of the blood vessel or the thickness of the vascular wall; however, by reinforcing the blood vessel by wrapping the tissue reinforcement materials around it, a higher power treatment in a shorter amount of time will be possible.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method for treating tissue, the method comprising:
applying a tissue reinforcement material to at least a portion of a tissue to be treated;
applying energy to the tissue reinforcement material, the tissue, or both the tissue reinforcement material and the tissue, wherein applying energy causes fusion between at least a part of the tissue reinforcement material and at least a part of the tissue; and
manipulating the tissue.

2. The method of claim 1, wherein manipulating the tissue comprises cutting the tissue.

3. The method of claim 2, wherein fusion occurs concurrently with cutting the tissue.

4. The method of claim 2, wherein fusion commences just before a commencement of cutting the tissue.

5. The method of claim 2, wherein fusion continues until after cutting the tissue.

6. The method of claim 2, wherein fusion occurs prior to cutting the tissue.

7. The method of claim 1, wherein manipulating the tissue comprises ligation.

8. The method of claim 1, wherein manipulating the tissue is performed by a device configured to impart energy to the tissue.

9. The method of claim 8, wherein the energy comprises heat.

10. The method of claim 8, wherein the energy comprises electricity.

11. The method of claim 8, wherein the energy comprises radiation.

12. The method of claim 1, wherein applying a tissue reinforcement material to at least a portion of the tissue comprises wrapping the tissue reinforcement material around a circumference of the tissue.

13. The method of claim 1, wherein the tissue reinforcement material comprises at least one of: an oxidized cellulose, a polyglycolic acid, a fibrinogen, collagen, or a lipid.

14. The method of claim 1, wherein applying energy comprises manipulating a temperature of the tissue reinforcement material.

15. The method of claim 1, wherein applying energy comprises controlling at least one of energy output, energy frequency, or both energy output and energy frequency.

16. The method of claim 1, wherein the energy comprises at least one of ultrasonic energy, electricity, light, microwave, radiation, or cryoablation.

17. The method of claim 1, wherein the fusion between at least a part of the tissue reinforcement material and at least a part of the tissue causes, at least partial hemostasis.

18. The method of claim 1, wherein the fusion is caused, at least in part, by denaturation of at least a part of the tissue.

19. The method of claim 18, wherein the denaturation occurs in a protein in the tissue.

20. The method of claim 1, wherein the tissue to be treated comprises at least one of a blood vessel, a neuron, a surface of an organ, or a muscle.

21. The method of claim 1, wherein the tissue reinforcement material comprises at least a lipid or lecithin.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A method for treating tissue, the method comprising:
applying a tissue reinforcement material to at least a portion of a tissue to be treated, wherein the tissue reinforcement material comprises a lipid; and
applying energy to the tissue reinforcement material, the tissue, or both the tissue reinforcement material and the tissue, wherein applying energy causes fusion between at least a part of the tissue reinforcement material and at least a part of the tissue.

50. The method of claim 49, wherein the tissue reinforcement material comprises one or more ingredients of lecithin.

51. The method of claim 49, wherein the reinforcement material comprises lecithin.

52. (canceled)

53. (canceled)

54. (canceled)
